(19) United States
(12) Patent Application Publication
(10) Pub. No.: US 2006/0142794 A1
(43) Pub. Date: Jun. 29, 2006

(54) Self-expanding device for the gastrointestinal or urogenital area

(75) Inventors: Andreas Lendlein, Berlin (DE); Robert S. Langer, Newton, MA (US)

Correspondence Address: PATREA L. PABST;PABST PATENT GROUP LLP, 400 COLONY SQUARE, SUITE 1200, ATLANTA, GA, 30361, US

(73) Assignee: Mnemoscience GmbH

(21) Appl. No.: 10/546,092

(22) PCT Filed: Feb. 18, 2004
(86) PCT No.: PCT/US04/04776

§ 371 Date: Dec. 16, 2005

Related U.S. Application Data
(60) Provisional application No. 60/448,568, filed on Feb. 19, 2003.

(51) Int. Cl. A61M 29/00

(52) U.S. Cl. 606/191

(57) ABSTRACT

Devices for treatments of diseases and disorders associated with the gastrointestinal tract, especially the stomach, or urinogenital tract are described herein. Initially, the device is in a temporary form which is suitable for oral or rectal administration. After exposure to a stimulus, such as a temperature or pH change, the device changes shape to a permanent form, which allows it to become mechanically fixed in the stomach, esophagus or intestine. In one embodiment, the device is used to reduce the volume of the stomach, esophagus or intestine without interfering with the flow of the food through the gastrointestinal tract. The device may be used to help overweight patients lose weight and to deliver drugs to treat disorders and diseases in the in the stomach or intestine. The devices are manufactured from a stimuli-sensitive polymeric material, which is biocompatible and primarily adapted to the mechanical properties and geometry in the area to which it is applied. In the preferred embodiment, the material is a shape memory polymer. Depending on the desired application, the polymer may be either biodegradable or non-degradable.

### FIELD OF THE INVENTION

The present invention relates to devices to treat diseases and disorders associated with the gastrointestinal or urinogenital area.

### BACKGROUND OF THE INVENTION

The transit time through the gastrointestinal (GI) tract often limits the amount of drug available for absorption at its most efficient absorption site, or for local activity at one segment of the GI tract. The latter is particularly true when the absorption site is high in the GI tract, for example, when the required treatment is local in the stomach as is often the case with ulcers.

A number of different patents describe oral compositions for increasing the time that the drug is delivered to the stomach. U.S. Pat. No. 4,451,260 to Mitra discloses orally administered, sustained release, flexible medicament devices which are formed from multilayer composites. These devices float in the stomach.

U.S. Pat. Nos. 4,735,804; 4,758,436; and 4,767,627 to Caldwell et al. disclose drug delivery devices that contain a polymeric, shaped solid that is retained in the stomach. The device is compressed for oral delivery, expands in the stomach to size that prevents passage through a pylorus, and then erodes over time in the presence of gastric juices. U.S. Pat. No. 5,007,790 to Shell describes as oral drug dosage form that swells upon delivery to the stomach so that it resides in the stomach and provides prolonged drug delivery. The drug is presented to the gastric mucosa as a solution, rather than in a solid state. U.S. Pat. No. 5,972,389 to Sheel discloses swellable polymer systems designed to deliver sparingly soluble or insoluble drugs into the gastrointestinal tract as a result of the gradual erosion of the polymer.

These compositions cannot be specifically designed to treat a variety of diseases and disorders.

Therefore it is an object of the invention to provide devices that can be tailored to treat different diseases and disorders of the gastrointestinal tract.

It is a further object of the invention to provide devices that can be easily removed from the gastrointestinal tract.

### BRIEF SUMMARY OF THE INVENTION

Devices for treatments of diseases and disorders associated with the gastrointestinal tract, especially the stomach, or urinogenital tract have been developed. Initially, the device is in a temporary form which is suitable for oral or intraluminal administration. After exposure to a stimulus, such as a temperature or pH change, the device changes shape to a permanent form, which allows it to become mechanically fixed in the stomach, esophagus or intestine. In one embodiment, the device is used to reduce the volume of the stomach, esophagus or intestine without interfering with the flow of the food through the gastrointestinal tract. The device may be used to help overweight patients lose weight and to deliver drugs to treat disorders and diseases in the in the stomach or intestine. The devices are manufactured from a stimuli-sensitive polymeric material, which is biocompatible and primarily adapted to the mechanical properties and geometry in the area to which it is applied. In the preferred embodiment, the material is a shape memory polymer. Depending on the desired application, the polymer may be either biodegradable or non-degradable.

### BRIEF DESCRIPTION OF THE DRAWINGS

FIG. 1 is a drawing of devices in their permanent forms.

FIG. 2 is a drawing of devices in their temporary forms. The temporary form may be compressed or elongated.

### DETAILED DESCRIPTION OF THE INVENTION

I. Devices

Devices for treatments of diseases and disorders associated with the gastrointestinal tract or urogenital region are described herein. The device has a form which allows it to become fixed mechanically, for example, either in the stomach, esophagus or intestine. The device is manufactured from a stimuli-sensitive polymeric material, which is biocompatible and primarily adapted to the mechanical properties and geometry in the area to which it is applied. In the preferred embodiment, the material is a shape-memory-polymer.

The device is capable of changing from one form to another form based on the presence of a stimulus. The stimulus may be a change in temperature or pH, or the presence/absence of water or light. The first form is referred to herein as the “temporary form”. The second form is referred to herein as the “permanent form”.

Different types of polymers respond to different stimuli. When the device is exposed to the appropriate stimulus, it changes shape (herein referred to as the “shape memory effect”). The shape memory effect is the transition from the temporary form to the permanent form. Suitable stimuli for activating the shape memory effect include: (1) an increase in temperature, (2) a change in the pH, (3) the application of light, and (4) the presence of water. The pH stimulus may be a change from a pH greater than 7 to one that is less than 7, such as occurs upon entry into the stomach. Alternatively, the pH stimulus may be from a pH that is less than 7 to one that is greater than 7, such as occurs upon entry into the intestine. Light may increase the temperature of the environment. Alternatively, light may catalyze a photosensitve or photochemical reaction in the material that forms the device. The presence of water may cause the device to swell and/or may increase diffusion of materials.

A. Shape Memory Polymers

Shape memory polymers (SMP) respond to a shape memory effect. Shape memory polymers are described in U.S. Pat. No. 6,160,084 to Langer et al., and U.S. Pat. No. 6,388,043 to Robert S. Langer and Andreas Lendlein, the disclosures of which are incorporated herein by reference.

SMPs are generally characterized as having netpoints and flexible segments. These netpoints can be chemical or physical in nature. SMPs are characterized as phase segregated linear block co-polymers having a hard segment and a soft segment. The hard segment is typically crystalline, with a defined melting point, and the soft segment is typically amorphous, with a defined glass transition temperature. In some embodiments, however, the hard segment is amorphous and has a glass transition temperature rather than a melting point. In other embodiments, the soft segment is crystalline and has a melting point rather than a glass transition temperature of the hard segment.

When the SMP is heated above the melting point or glass transition temperature of the hard segment, the material can be shaped. This permanent or original shape can be memorized by cooling the SMP below the melting point or glass transition temperature of the hard segment. When the shaped SMP is cooled below the melting point or glass transition temperature of the soft segment white the shape is deformed, a new (temporary) shape is fixed. The original shape is recovered by heating the material above the melting point or glass transition temperature of the soft segment but below the melting point or glass transition temperature of the hard segment. In another method for setting a temporary shape, the material is deformed at a temperature lower than the melting point or glass transition temperature of the soft segment, resulting in stress and strain being absorbed by the soft segment. When the material is heated above the melting point or glass transition temperature of the soft segment, but below the melting point (or glass transition temperature) of the hard segment, the stresses and strains are relieved and the material returns to its original shape. The recovery of the original shape, which is induced by an increase in temperature, is called the thermal shape memory effect. Properties that describe the shape memory capabilities of a material are the shape recovery of the original shape and the shape fixity of the temporary shape.

Several physical properties of SMPs other than the ability to memorize shape are significantly altered in response to external changes in temperature and stress, particularly at the melting point or glass transition temperature of the soft segment. These properties include the elastic modulus, hardness, flexibility, vapor permeability, damping, index of refraction, and dielectric constant. The elastic modulus (the ratio of the stress in a body to the corresponding strain) of an SMP can change by a factor of up to 200 when heated above the melting point or glass transition temperature of the soft segment. Also, the hardness of the material changes dramatically when the soft segment is at or above its melting point or glass transition temperature. When the material is heated to a temperature above the melting point or glass transition temperature of the soft segment, the damping ability can be up to five times higher than a conventional rubber product. The material can readily recover to its original molded shape following numerous thermal cycles, and can be heated above the melting point of the hard segment and reshaped and cooled to fix a new original shape.

Preferred SMPs can hold more than one shape in memory. For example, the composition can include a hard segment and at least two soft segments. The Tₜᵣₐₙₛ of the hard segment is at least 10° C., and preferably 20° C., higher than the Tₜᵣₐₙₛ of one of the soft segments, and the Tₜᵣₐₙₛ of each subsequent soft segment is at least 10° C., and preferably 20° C., lower than the Tₜᵣₐₙₛ of the preceding soft segment. A multiblock copolymer with a hard segment with a relatively high Tₜᵣₐₙₛ and a soft segment with a relatively low Tₜᵣₐₙₛ can be mixed or blended with a second multiblock copolymer with a hard segment with a relatively low Tₜᵣₐₙₛ and the same soft segment as that in the first multiblock copolymer. Since the soft segments in both multiblock copolymers are identical, the polymers are miscible in each other when the soft segments are melted. The resulting blend has three transition temperatures: one for the first hard segment, one for the second hard segment, and one for the soft segment. Accordingly, these materials are able to memorize two different shapes.

The hard segments can be linear oligomers or polymers, and can be cyclic compounds, such as crown ethers, cyclic di-, tri-, or oligopetides, and cyclic oligo (ester amides).

The physical interaction between hard segments can be based on charge transfer complexes, hydrogen bonds, or other interactions, since some segments have melting temperatures that are higher than the degradation temperature. In these cases, there is no melting or glass transition temperature for the segment. A non-thermal mechanism, such as a solvent, is required to change the segment bonding.

The segments preferably are oligomers. As used herein; the term “oligomers” refers to a linear chain molecule having a molecular weight up to 15,000 Da. The ratio by weight of the hard segment: soft segments is between about 5:95 and 95:5, preferably between 20:80 and 80:20.

The polymers are selected based on the desired glass transition temperature(s) (if at least one segment is amorphous) or the melting point(s) (if at least one segment is crystalline), which in turn is based on the desired applications, taking into consideration the environment of use. Preferably, the number average molecular weight of the polymer block is greater than 400, and is preferably in the range of between 500 and 15,000.

The transition temperature at which the polymer abruptly becomes soft and deforms can be controlled by changing the monomer composition and the kind of monomer, which enables one to adjust the shape memory effect at a desired temperature.

The thermal properties of the polymers can be detected for example, by dynamic mechanical thermoanalysis or differential scanning calorimetry (DSC) studies. In addition the melting point can be determined using a standard mp apparatus.

The polymers can be thermoset or thermoplastic polymers, although thermoplastic polymers may be preferred due to their ease of molding.

Preferably, the degree of crystallinity of the polymer or polymeric block(s) is between 3 and 80%, more preferably between 3 and 60%. When the degree of crystalinity is greater than 80% while all soft segments are amorphous, the resulting polymer composition has poor shape memory characteristics.

The tensile modulus of the polymers below the Tₜᵣₐₙₛ is typically between 50 MPa and 2 GPa (gigapascals), whereas the tensile modulus of the polymers above the Tₜᵣₐₙₛ is typically between 1 and 500 MPa. Preferably, the ratio of elastic modulus above and below the Tₜᵣₐₙₛ is 20 or more. The higher the ratio, the better the shape memory of the resulting polymer composition.

The polymer segments can be natural or synthetic, although synthetic polymers are preferred. The polymer segments can be biodegradable or non-biodegradable, although the resulting SMP composition is biodegradable. As used herein, the term “biodegradable” typically refers to materials that are bioresorbable and/or degrade and/or break down by mechanical degradation upon interaction with a physiological environment into components that are metabolizable or excretable, over a period of time from minutes to three years, preferably less than one year, while maintaining the requisite structural integrity. In general, biodegradable materials degrade by hydrolysis, by exposure to water or enzymes under physiological conditions, by surface erosion, bulk erosion, or a combination thereof. Non-biodegradable polymers used for medical applications preferably do not include aromatic groups, other than those present in naturally occurring amino acids.

Representative natural polymer segments or polymers include proteins such as zein, modified zein, casein, gelatin, gluten, serum albumin, and collagen, and polysaccharides such as alginate, celluloses, dextrans, pullulane, and polyhyaluronic acid, as well as chitin, poly(3-hydroxyalkanoate)s, especially poly(β-hydroxybutyrate), poly(3-hydroxyoctanoate) and poly(3-hydroxyfatty acids).

Representative natural biodegradable polymer segments or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Representative synthetic polymer blocks include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters polyvinyl ethers, polyvinyl esters, polyvinyl halides, polyvinylpyrrolidone, polyesters, polylactides, polyglycolides, polysiloxanes, polyurethanes and copolymers thereof.

Examples of suitable polyacrylates include poly(methyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxyporpyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. These are collectively referred to herein as “celluloses”.

Representative synthetic degradable polymer segments or polymers include polyhydroxy acids, such as polylactides, polyglycolides and copolymers thereof; poly(ethylene terephthalate); polyhydroxybutyric acid); poly(hydroxyvaleric acid); poly([lactide-co-(ε-caprolactone)]; poly[glycolide-co-(ε-caprolactone)]; polycarbonates, poly(pseudo amino acids); poly(amino acids); poly(hydroxyalkanoate)s; polyanhydrides; polyortho esters; and blends and copolymers thereof.

Examples of non-biodegradable polymer segments or polymers include ethylene vinyl acetate, poly(meth)acrylic acid, polyamides, polyethylene, polypropylene, polystyrene, polyvinyl chloride, polyvinylphenol, and copolymers and mixtures thereof.

Rapidly bioerodible polymers such as poly(lactide-co-glycolide)s, polyanhydrides, and polyorthoesters, which have carboxylic groups exposed on the external surface as the smooth surface of the polymer erodes, also can be used. In addition, polymers containing labile bonds, such as polyanhydrides and polyesters, are well known for their hydrolytic reactivity. Their hydrolytic degradation rates can generally be altered by simple changes in the polymer backbone and their sequence structure.

Various polymers, such as polyacetylene and polypyrrole, are conducting polymers. These materials are particularly preferred for uses in which electrical conductance is important. Examples of these uses include tissue engineering and any biomedical application where cell growth is to be stimulated. These materials may find particular utility in the field of computer science, as they are able to absorb heat without increasing in temperature better than SMAs. Conducting shape memory polymers are useful in the field of tissue engineering to stimulate the growth of tissue, for example, nerve tissue.

Shape memory polymers that are generally usable include crystalline polyolefin crosslinked substances, crystalline trans-isoprene crosslinked substances, crystalline trans-polybutadiene crosslinked substances, polynorbornene, poly(vinylchloride), poly(methyl methacrylate), polycarbonate, acrylonitrile-butadiene (AB) resin, polyethers, polyamides, polysiloxanes, polyurethanes, polyether amides, polyurethane/ureas, polyether esters, and urethane/butadiene copolymers.

The shape memory effect can also be triggered by contact of the shape memory polymer (SMP) with water. This SMP is characterized by a glass transition temperature and is preferably amorphous. The programming of the SMP can be carried out using standard thermal shape memory. The polymer is able to absorb a certain amount of water like a hydrogel, however, the resulting degree of swelling is smaller, for example, the weight of the SMP increases about 0.5 to 4 wt %. Compared to a hydrogel, the mechanical properties of this slightly swollen: material are mainly like the, bulk material (non-swollen). The absorption of water leads to a decrease in glass transition temperature of about 10 to 30 K (softening effect). Therefore, a glass transition temperature which was originally above body temperature can be decreased to below body temperature. When such an SMP is used at body temperature, in the stomach, for example, the shape memory effect will be activated (by water absorption). The swelling of the SMP occurs preferably within 20 to 90 minutes and should correspond with the residence time of the SMP device in the stomach, which is, typically 2 to 4 hours.

In addition, the swelling of the SMP can be altered by adjusting the pH and/or by coating the SMP with a pH-sensitive material so that swelling only occurs at certain pH ranges. For example, pH-sensitive coatings, which are well-known in the pharmaceutical industry; can be used to allow swelling of the SMP only at a lower pH, for applications in the stomach, or at a higher pH, for applications in the intestinal tract. Thus, pH-sensitive coatings can be used to prevent swelling of the SMP in the esophagus when, delivered orally.

Typically the pH-sensitive materials are insoluble solids in neutral or acidic aqueous solutions, and then they dissolve (or degrade and dissolve) as the pH of the solution rises above a pH value ranging from 3 to 9, preferably 6 to 8. Exemplary pH-sensitive materials include polyacrylamides, phthalate derivatives (i.e., compounds with covalently attached phthalate moleties) such as acid phthalates of carbohydrates, amylose acetate phthalate, cellulose acetate phthalate, other cellulose ester phthalates, cellulose ether phthalates, hydroxypropyl cellulose phthalate, hydroxypropyl ethylcellulose phthalate, hydroxypropyl methyl cellulose phthalate, methyl cellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinyl acetate phthalate copolymer, styrene and maleic acid copolymers, formalized gelatin, gluten, shellac, salol, keratin, keratin sandarac-tolu, ammoniated shellac, benzophenyl salicylate, cellulose acetate trimellitate, cellulose acetate blended with shellac, hydroxypropylmethyl cellulose acetate succinate, oxidized cellulose, polyacrylic acid derivatives such as acrylic acid and acrylic ester copolymers, methacrylic acid and esters thereof, vinyl acetate and crotonic acid copolymers.

Preferred pH-sensitive materials include shellac; phthalate derivatives, particularly cellulose acetate phthalate, polyvinyl acetate phthalate and hydroxypropyl methylcellulose phthalate; polyacrylic acid derivatives, particularly polymethyl methacrylate blended with acrylic acid and acrylic ester copolymers; and vinyl acetate and crotonic acid copolymers.

The pH-sensitive material is preferably blended with an inert non-dissolving material. By inert is meant a material that is not substantially affected by a change in pH in the triggering range. By altering the proportion of a pH-sensitive material to inert non-dissolving material the time lag subsequent to triggering and prior to release may be tailored. For example, for capsule devices, the blend of pH-sensitive material to inert non-dissolving material may be tailored to control the time when the capsule halves separate after being triggered. Thus, preferably a proportional mixture of pH-sensitive material to inert nondissolving material is used that provides the desired release time lag subsequent to triggering. Any inert non-dissolving material may be used that does not react with the trigger. Typically, increasing the proportion of inert nondissolving material will lengthen the time lag after triggering and subsequent to release of the beneficial agent. Preferably, the inert material is selected from the list of materials given for the semipermeable membrane (above).

Alternatively pH-sensitive materials can be used that are insoluble solids in neutral or alkaline solutions, and then they dissolve (or degrade and dissolve) as the pH of the solution drops below a pH value ranging from 3 to 9. Exemplary pH-sensitive materials include copolymers of acrylate polymers with amino substituents and acrylic acid esters. Additional pH-sensitive materials include polyfunctional polymers containing multiple groups that become ionized as the pH drops below their pKa. A sufficient quantity of these ionizable groups must be incorporated in the polymer such that in aqueous solutions having a pH below the pKa of the ionizable groups, the polymer dissolves. These ionizable groups can be incorporated into polymers as block copolymers, or can be pendent groups attached to a polymer backbone, or can be a portion of a material used to crosslink or connect polymer chains. Examples of such ionizable groups include polyphosphene, vinyl pyridine, vinyl aniline, polylysine, polyornithine, other proteins, and polymers with substituents containing amino moieties.

In one embodiment, the programmable SMP has a thermal shape memory and is able to swell in an aqueous medium like a hydrogel. The polymer may optionally be ionically cross-linked with multivalent ions or polymers. When the programmed polymer swells, the physical crosslinks disappear and trigger the shape memory effect. In contrast to hydrogels, the shape changes and the volume increases in the SMP.

In another embodiment, the swelling of the SMP can be adjusted by altering the pH, and in a preferred embodiment, the SMP comprises a pH sensitive coating which allows swelling only at specific pH ranges.

The polymer may also be in the form of a hydrogel (typically absorbing up to about 90% by weight of water), and can optionally be ionically crosslinked with multivalent ions or polymers. Ionic crosslinking between soft segments can be used to hold a structure, which when deformed, can be reformed by breaking the ionic crosslinks between the soft segments. The polymer may also be in the form of a gel in solvents other than water or aqueous solutions. In these polymers, the temporary shape can be fixed by hydrophilic interactions between soft segments.

Hydrogels can be formed from polyethylene glycol, polyethylene oxide, polyvinyl alcohol, polyvinyl pyrrolidone, polyacrylates, poly(ethylene terephthalate), poly(vinyl acetate), and copolymers and blends thereof. Several polymeric segments, for example, acrylic acid, are elastomeric only when the polymer is hydrated and hydrogels are formed. Other polymeric segments, for example, methacrylic acid, are crystalline and capable of melting even when the polymers are not hydrated. Either type of polymeric block can be used, depending on the desired application and conditions of use.

For example, shape memory is observed for acrylic acid copolymers only in the hydrogel state, because of the acrylic acid units are substantially hydrated and behave like a soft elastomer with a very low glass transition temperature. The dry polymers are not shape memory polymers. When dry, the acrylic acid units behave as a hard plastic even above the glass transition temperature and show no abrupt change in mechanical properties on heating. In contrast, copolymers including methyl acrylate polymeric segments as the soft segments show shape memory properties even when dry.

The polymers can be obtained from commercial sources such as Sigma Chemical Co., St. Louis, Mo.; Polysciences, Warrenton, Pa.; Aldrich Chemical Co., Milwaukee, Wis.; Fluka, Ronkonkoma, N.Y.; and BioRad, Richmond, Calif. Alternately, the polymers can be synthesized from monomers obtained from commercial sources, using standard techniques.

B. Forms of the Devices

In the temporary form, the device is fixed in a compressed or stretched shape due to the shape memory effect of the matrix material (see FIG. 2). The shape of the device in its temporary form is selected so that the device is suitable for swallowing by a patient or for rectal or urinogenital administration. In this situation, the shape will be determined by the application, for example, for gastric reduction, the size of the device will be based on how much stomach is to be filled by the device.

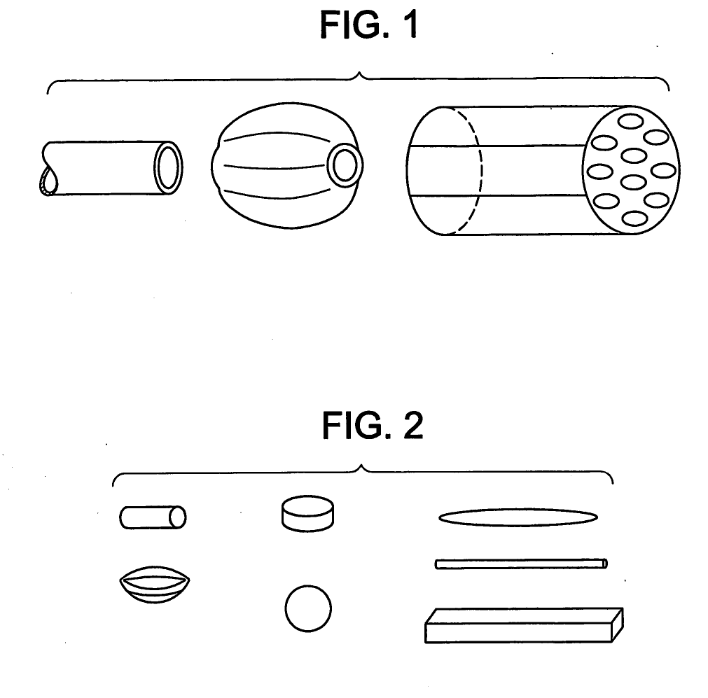

After exposure to the stimulus, the device changes to a permanent form (see FIG. 1). The permanent form fixes mechanically in the stomach, esophagus or intestine. In the preferred embodiment, the device is used for gastric reduction. The device reduces the volume of the stomach, esophagus or intestine without interfering with the flow of the food through the gastrointestinal tract. The reduction in volume may be great or small. For example, in the case of an anorectic device which is used to assist in weight loss, a large volume of the stomach should be filled with the device. In contrast, when the device serves as a drug depot, delivery device for biologically active agents, or as a protective coating, the reduction in the volume in the stomach, esophagus or intestine should be minimal. Overweight patients can use the device to lose weight. The device fills the stomach thereby reducing the capacity of the stomach for food and the feeling of hunger.

In another embodiment, the device is a matrix used in the treatment of gastritis. The matrix lines the stomach's septum and thereby protects the stomach against the contents or juice of the stomach for a discrete period of time.

1. Drug Delivery

For drug delivery, device may be in the form of a pill or capsule (see FIG. 2). Alternatively, the device may be incorporated in a capsule. However, in this embodiment, the capsule does not serve as the temporary form. Typically, the device is loaded with one or more biologically active agents, including drugs, prophylactics or diagnostic or analytical agents (e.g. contrast medium). These may be organic compounds, proteins or peptides, sugars or carbohydrates, nucleic acids, lipids, or combinations thereof. The material of the device can be biodegradable or non-biodegradable. Optionally, the device is coated to improve its shelf-life, increase slippage for swallowing, or improve the general infiltration into the stomach or intestine, or alter release characteristics.

In one embodiment, the device is a matrix that forms a stent-like device in the esophagus. The matrix may contain one or more biologically active agents, such as drugs. For example, the drug may be effective in the treatment of pyrosis.

2. Urogenital Devices

The device may be suitable for administration to the urinogenital tract. Optionally, the device contains one or more biologically active agents. In one embodiment, the device acts as a contraceptive. For urogenital applications, for example, for contraception or drug delivery in the uterus, the device will be shaped for ease of insertion into the vagina or cervix, where it enlarges or alters shape so that it is retained. For bladder disorders, such as reflux or incontinence, the device is shaped so that it can be safely positioned at a point where additional retention is desired, such as the point at which the ureter connects to the bladder.

C. Biologically Active Agents

The device may contain one or more biologically active agents, such as drugs and diagnostic agents, which are effective at treating disorders and diseases in the gastrointestinal tract. The term “drug” refers to any pharmaceutically active substance capable of being administered in a particulate formulation, which achieves the desired effect. Drugs can be synthetic or natural organic compounds, proteins or peptides, oligonucleotides or nucleotides, or polysaccharides or sugars. Drugs may have any of a variety of activities, which may be inhibitory or stimulatory, such as antibiotic activity, antiviral activity, antifungal activity, steroidal activity, cytotoxic or anti-proliferative activity, anti-inflammatory activity, analgesic or anesthetic activity, or be useful as contrast or other diagnostic agents. A description of classes of drugs and species within each class can be found in Martindale, The Extra Pharmacopoeia, 31^{st} Ed., The Pharmaceutical Press, London (1996) and Goodman and Gilman, The Pharmacological Basis of Therapeutics, (9^{th} Ed., McGraw-Hill Publishing company (1996)). In a preferred embodiment, the agent is suitable for treating disorders and diseases in the stomach or intestine, including but not limited to gastritis, gastroparesis, peptic ulcers, Menetrier's disease and gastric and colorectal cancer. In another embodiment, the agent is used for treatment of urogenital infections and disorders including but not limited to bacterial vaginosis, trichomoniasis, candidiasis, ovarian cancer, vaginal cancer, cervical cancer, prostate cancer, bladder cancer, kidney cancer, vulvar cancer, uterine cancer, urinary tract infections, and incontinence. Finally, the agent may also be used for contraception.

II. Methods of Making the Devices

The devices can be formed by standard techniques to mold, cast or shape the device.

The devices can be prepared using shape memory polymers. In one embodiment, the SMP contains a hard segment, a first soft segment, and a second soft segment, where the first soft segment has a Tₜᵣₐₙₛ at least 10° C. below that of the hard segment and at least 10° C. above that of the second soft segment. After the composition is shaped at a temperature above the Tₜᵣₐₙₛ of the hard segment it can be cooled to a temperature below that of the Tₜᵣₐₙₛ of the first soft segment and above that of the second soft segment and formed into a second shape. The composition can be formed into a third shape after it has been cooled below the Tₜᵣₐₙₛ of the second soft segment. The composition can be heated above the second soft segment to return the composition to the second shape. The composition can be heated above the Tₜᵣₐₙₛ of the first soft segment to return the composition to the first shape. The composition can also be heated above the Tₜᵣₐₙₛ of the hard segment, at which point the composition loses the memory of the first and second shapes and can be reshaped using the method described above.

III. Methods of Using the Devices

The device can be delivered orally to a patient for delivery to the gastrointestinal tract. Alternatively, the device can be administered rectally for treatment of the gastrointestinal tract. Typically the device would be administered through the vagina or ureter to the urinogenital tract. One or several devices can be applied at the same time. After the device has remained at the site in the gastrointestinal tract for the prescribed period of time, it is expelled from the site.

In one embodiment, the material is hydrolytically or enzymatically degradable within a predetermined period of time. Soluble products of decomposition or intestine moving particles are then secreted.

In a second embodiment, the material returns to the first temporary form or to a second programmed temporary form, which is so small that the device is not longer mechanically fixed to the site and the device is secreted. Stimuli for the transition from the permanent form: to the first or a second temporary form include: (1) a change in temperature, (2) a substance that delivers the stimuli by taking it at any point of time, (3) light, e.g. ultraviolet or infrared, and (4) ultrasound.

It is understood that the disclosed invention is not limited to the particular methodology, protocols, and reagents described as these may vary. It is also to be understood that the terminology used herein is for the purpose of describing particular embodiments only and is not intended to limit the scope of the present invention which will be limited only by the appended claims.

1. A device to treat diseases and disorders of the gastrointestinal or urinogenital tract comprising a shape memory polymer having a first temporary form allowing the device to be inserted and a second permanent form to retain the device within the gastrointestinal or urogenital tract.

2. The device in claim 1, further comprising a biologically active agent.

3. The device in claim 1, further comprising a pH sensitive coating.

4. The device in claim 1, wherein the shape memory polymer is biodegradable.

5. The device of claim 1, wherein the shape memory polymer changes from the first form to the second form upon exposure to a stimulus.

6. The device of claim 1 for gastric retention.

7. The device of claim 1 for insertion into the uterus via the vagina.

8. The device of claim 1 for insertion into the ureter.

9. A method for treating diseases and disorders of the gastrointestinal tract or urogenital tract comprising orally administering a composition to a patient, wherein the composition comprises a device comprising a shape memory polymer, and exposing the device to a stimulus which changes the form of the device.

10. The method of claim 9, wherein the composition is delivered to the stomach.

11. The method of claim 9, wherein the composition is delivered to the esophagus.

12. The method of claim 9, wherein the stimulus is selected from the group consisting of a change in temperature, a change in the pH, light, and water.

13. The device of claim 5 wherein the stimulus is selected from the group consisting of a change in temperature, a change in the pH, light, and water.
